(19) United States
(12) Patent Application Publication
(10) Pub. No.: US 2004/0191801 A1
(43) Pub. Date: Sep. 30, 2004

(54) Reagentless, reusable bioelectronic detectors and their use as authentication devices

(76) Inventors: Alan J. Heeger, Santa Barbara, CA (US); Chunhai Fan, Shanghai (CN); Kevin Plaxco, Santa Barbara, CA (US)

Correspondence Address: William H. Benz, Foley & Lardner LLP, Three Palo Alto Square, 3000 El Camino Real, Suite 100, Palo Alto, CA, 94306-2121, US

(21) Appl. No.: 10/678,760

(22) Filed: Oct. 3, 2003

Related U.S. Application Data
(60) Provisional application No. 60/457,762, filed on Mar. 25, 2003.

(51) Int. Cl. C12Q 1/68; C12M 1/34

(52) U.S. Cl. 435/6; 435/287.2; 205/777.5

(57) ABSTRACT

A reagentless, reusable bioelectronic DNA or RNA sequence sensor is disclosed. The sensor includes a DNA probe tagged with a electroactive, redoxable moiety, self-assembled on or near an electrode. This surface-confined DNA probe structure undergoes hybridization-induced conformational change in the presence of the target DNA/RNA sequence which change the electron-transfer distance between the redoxable moiety and the electrode thereby providing a detectable signal change. In a preferred application, the target sequence is associated with an object and its detection is correlated with the authenticity of the object.

### REFERENCE TO RELATED APPLICATIONS

This application is related to and claims the benefit of U.S. Provisional Application Serial No. 60/457,762 filed on Mar. 25, 2003.

### REFERENCE TO GOVERNMENT SUPPORT

This invention was made in part with government support under grants from the National Science Foundation, (Grant No. NSF-DMR-0099843), the Office of Naval Research Grant (ONR N0014-1-1-0239) and the National Institute of Health Grant (Grant No. GM 62958-01).

### BACKGROUND OF THE INVENTION

1. Field of the Invention

This invention relates to bioelectronic sensors and their use to detect hybridization events occurring in DNA and RNA systems. In a preferred embodiment the detection of such hybridization events is used to detect and verify a DNA authentication tag.

2. Background Information

The detection of DNA/RNA (hereinafter generally “DNA”) hybridization events is of significant scientific and technological importance, manifested in, for example, the rapidly growing interest in the chip-based characterization of gene expression patterns and the detection of pathogens in both clinical and civil defense settings [Heller, M. J., Annu. Rev. Biomed. Eng. 4, 129-153 (2002)]. Consequently, a variety of optical[Taton, T. A., Mirkin, C. A. & Letsinger, R. L. Science 289, 1757-1760 (2000); Gaylord, B. S., Heeger, A. J. & Bazan, G. C., Proc. Nat. Acad. Sci. USA 99, 10954 (2002); Cao, Y. W. C., Jin, R. C. & Mirkin, C. A., Science 297, 1536-1540 (2002)] and acoustic [Cooper, M. A. et al. Nature Biotech. 19, 833-837 (2001) detection methods have been proposed.

In these assays one or more target polynucleotides is brought into proximity to one or more polynucleotide ligands and hybridization (if any) is detected by noting a change in a detectable “genosensor” moiety such as the presence of a suitable fluorolabel, radiolabel or enzyme label present on the ligands.

Among these historic genosensors, fluorescence detection methods have historically dominated the state of the art [Heller, M. J., Annu. Rev. Biomed. Eng. 4, 129-153 (2002); Bowtell, D. D. L., Nature Genet. 21, 25-32 (1999); Winzeler, E. A., Schena, M. & Davis, R. W., Methods Enzymol. 306, 3 (1999)].

The application of electronic methods to the sensing of biologically related species, however, has attracted rapidly increasing attention [Kuhr, W. G., Nature Biotech. 18, 1042-1043 (2000); Willner, I., Science 298, 2407 (2002); Fritz, J., Cooper, E. B., Gaudet, S., Sorger, P. K. & Manalis, S. R. Electronic detection of DNA by its intrinsic molecular charge. Proc. Natl. Acad. Sci., U.S.A. 99, 14142-14146 (2002)].

Advantages of bioelectronic detection include the following:

1. Electrochemical techniques offer the promise of sensitive, rapid and inexpensive screening [Bard, A. J. & Faulkner, L. R. Electrochemical Methods (John W. Willey & Sons, New York, 2001)].

2. Unlike fluorophores that quench or photo-bleach, typical electroactive labels are stable and relatively insensitive to their environment.

3. “Multi-color” labeling is possible by molecular design and synthesis that produce a “spectrum” of derivatives, each having a unique detectable electronic signal [Brazill, S. A., Kim, P. H. & Kuhr, W. G., Anal. Chem. 73, 4882-4890 (2001)].

4. The possibility of mass-production of bioelectronic detectors via the well-developed technical infrastructure of the microelectronics industry, renders electronic detection particularly compatible with microarray-based technologies.

DNA itself is electrochemically silent at moderate applied voltages [Palecek, E. & Jelen, F., Crit. Rev. Anal. Chem. 32, 261-270 (2002)]. The first sequence-selective electronic method for DNA detection was based on the electrochemical interrogation of redox-active intercolators that bind preferably to double-stranded DNA [Millan, K. M. & Mikkelsen, S. R., Anal. Chem. 65, 2317-2323 (1993)]. More recently, the sensitivity of this detection approach was improved via electrocatalytic amplification [Kelley, S. O., Boon, E. M., Barton, J. K. & Jackson, N. M. H., M. G.;. Nucleic Acids Res. 27, 4830-4837 (1999)].

In an attempt to reduce high background deriving from the inappropriate binding of hybridization indicators to ssDNA, a “sandwich” type detector has been developed. This approach utilizes an electrode-attached ssDNA sequence that binds the target to the electrode and a second, redox-labeled ligand sequence complimentary to an adjacent sequence on the target [Ihara, T., Maruo, Y., Takenaka, S. & Takagi, M., Nucleic Acids Res. 24, 4273-4280 (1996); Yu, C. J. et al., J. Am. Chem. Soc. 123, 11155-11161 (2001); Umek, R. M. et al., J. Mol. Diag. 3, 74-84 (2001)].

Mirkin and co-workers have developed an electronic DNA detection approach that has demonstrated high sensitivity and selectivity [Park, S. J., Taton, T. A. & Mirkin, C. A,. Science 295, 1503-1506 (2002)]. In this resistance-based method, a probe-captured target undergoes a second hybridization event with Au nanoparticle-labeled DNA strands. Subsequent catalytic deposition of silver onto the Au nanoparticles leads to electrical contact and a detectable decrease in the resistance between electrode pairs as an indicator of hybridization.

Despite this interest in electronic DNA detection, there has been little progress toward the important goal of creating a sensor that is simultaneously sensitive, selective and reagentless (That is a sensor obviating further treatment with either hybridization indicators or signalling molecules to yield a detectable indication of hybridization). The “reagentless” feature has been reported in the context of a conjugated polymer-based electrochemical DNA sensor [Korri-Youssoufi, H., Garnier, F., Srivastava, P., Godillot, P. & Yassar, A., J. Am. Chem. Soc. 119, 7388-7389 (1997)]. However, this sensor has only moderate sensitivity due to broad, weakly-defined redox peaks.

More generally, while sensitivity of electronic DNA sensors of the prior art is impressive (ranging from 0.5 to 32 pM), no electronic sensors have been reported to meet the goal of fM sensitivity. The sensitive sensors require the addition of one or more exogenous reagents.

The present invention provides a system that uses these electrochemical DNA (E-DNA) sensors to detect and verify a DNA authentication tag. The technologies underlying counterfeiting generally keep pace with the technologies aimed at impeding such efforts and thus, to date, no general, unbreakable means of “authenticating” documents, drugs and other high-volume materials has been reported.

Recent, high profile examples ranging from geopolitical (e.g. forged documents purporting the solicitation of yellow-cake sales to Iraq) to the medical (e.g. the recent recall of approximately 100,000 bottles of potentially counterfeit Lipitor tablets) are indicative of the growing and increasingly complex risks associated with the counterfeiting of a wide range of documents and materials. Thus motivated, significant research has focused on the development of convenient-yet-unforgeable means of “authentifying” the provenance of documents, drugs and other materials related to medical, industrial, homeland or military security.

The use of DNA as an identifying label was first proposed by Philippe Labacq in U.S. Pat. No. 5,139,812 in 1992. The approach works by concealing coded messages in DNA. Security is provided by the inherent sequence complexity of DNA (Clelland, C. T., Risca, V. & Bancroft, C. Nature 399, 533-534 (1999)).

Existing DNA-based authentication methods, however, have been limited to art, sports memorabilia and other high-value, low-volume applications. More widespread use of the approach has been limited by the cumbersome, time and reagent-intensive methods currently employed for the detection of low concentrations of a target DNA sequence in the presence of orders of magnitude larger background of masking DNA (Clelland, C. T., Risca, V. & Bancroft, C. Nature 399, 533-534 (1999); Cox, J. P. L. Analyst 126, 545-547 (2001)).

It is the object of this invention to provide an electrochemical method for detecting specific sequences on target DNA, said method being simultaneously sensitive, selective, reagentless, and reusable. It is a further object to provide an electrochemical method for detecting a DNA authentication tag.

### STATEMENT OF THE INVENTION

We have now discovered a detector and system for determining the presence of a target polynucleotide having a target nucleotide sequence. The detector has an electrode capable of sensing redox events in a redox moiety and an immobilized polynucleotide probe which carries a redox moiety and has a probe nucleotide sequence which hybridizes with the target nucleotide sequence. The probe has a first configuration, in the absence of hybridization with the target polynucleotide, which locates the redox moiety in a first position relative to the electrode. The probe has a second configuration in the presence of hybridization with the target polynucleotide, which locates the redox moiety in a second position relative to the electrode. The first and second positions give rise to distinguishable redox events that are detectable by the electrode.

The first position may be closer to the electrode than the second position or vice versa.

In presently preferred embodiments, the probe is immobilized on the electrode.

In some preferred embodiments one or both of the first and second configurations may include a stem and hairpin (stem and loop) configuration with the stem immobilized on the electrode and with the redox moiety attached to the end of the polynucleotide probe distall from the stem.

In a second aspect, this invention concerns a method for detecting the presence of a target polynucleotide having a target nucleotide sequence in a sample. This method involves contacting the sample under polynucleotide hybridization conditions with the detector just described and sensing redox events in the redox moiety in the presence of the sample and redox events with the detector in the absence of the sample and correlating similarity in redox events between the two sensings with the absence of the target polynucleotide and a change in redox events with the presence of the target polynucleotide.

In a third aspect this invention provides a rapid, reagentless, E-DNA process for convenient, secure and inexpensive authentication. The E-DNA approach unambiguously determines the provenance of materials via the sequence specific detection of nanogram quantities of a DNA-based authentication tag. A many-fold excess of non-cognate, “masking DNA,” which may be included in order to thwart efforts to forge the authentication tag via cloning or sequencing, does not detectably alter the authentication signal. Using an inexpensive electrochemical workstation, robust authentication signals are obtained via salt-water extraction of authentication tags from dried paper, via the dissolution of a solid, orally-ingested drug and from small aliquot parts of an injectable drug all within ˜10 minutes and without further processing or the addition of exogenous reagents.

### DETAILED DESCRIPTION OF THE INVENTION

### Brief Description of the Drawings

This invention will be further described with reference being made to the drawings in which:

FIG. 1 is a not-to-scale diagram illustrating the mechanism by which a detector of this invention generates an indication of a DNA hybridization event. In this embodiment, the detector provides a decrease in signal as a measure of hybridization.

FIG. 2 is a second not-to-scale diagram illustrating the mechanism by which a second embodiment of the detector of the invention provides an increase in signal as a measure of hybridization.

FIG. 3 is a third not-to-scale diagram illustrating a third mechanism by which a third embodiment of the detector of the invention provides an indication of hybridization.

FIG. 4 is a fourth not-to-scale diagram illustrating two additional mechanisms by which additional embodiments of the detector of the invention provides an indication of hybridization.

FIG. 5a is a cyclic voltammogram for a gold electrode modified with the ferrocene tagged stem-loop oligonucleotide in a 1 M NaClO₄ solution, at a scan rate of 0.1 V/s. FIG. 5b demonstrates the relationship between the peak current and the scan rate.

FIG. 6a is a series of background-subtracted [Fan, C., Gillespie, B., Wang, G., Heeger, A. J. & Plaxco, K. W., J. Phys. Chem. (B) 106, 11375-11383 (2002).Hirst, J. et al. J. Am. Chem. Soc. 120, 7085-7094 (1998)] voltammograms (anodic scan) for a hairpin DNA modified gold electrode in the presence of complementary DNA at different concentrations: 0, 30 pM, 500 pM, 30 nM, 800 nM, 5 uM (from bottom to top). The hybridization was performed in a 1 M NaClO₄ solution, and the hybridization time was fixed at 30 min. FIG. 6bis a calibration curve (peak height vs. concentration of the complementary DNA).

FIG. 7 is a graph illustrating that at a target concentration of 500 pM, the signal develops in minutes. At this target concentration, the signal change observed after one hour of hybridzation implies that 65% of the probe-DNA has been hybridized by complementary ssDNA (at 5 mM the signal goes to zero within 30 minutes).

FIG. 8 is a cyclic voltammogram for a gold electrode modified with a methylene blue-tagged oligonucleotide in the absence of target DNA.

FIG. 9 is a series of AC voltammograms for the E-DNA sensor before a test (upper line) and after a test with DNA microdots containing masking DNA only (lower line) and masking DNA with target (upper line).

FIG. 10 is a graphic comparison among the E-DNA authentication signals observed before and after counterfeiting tests on three possible counterfitted objects.

FIG. 11 is a graphic comparison among E-DNA authentication signals generated in essentially the same manner as the signals in FIG. 10 with the addition of glycerol as an additive to reduce background noise. This figure displays the amount of signal change that was observed.

### DESCRIPTION OF PREFERRED EMBODIMENTS

Representative E-DNA Sensors

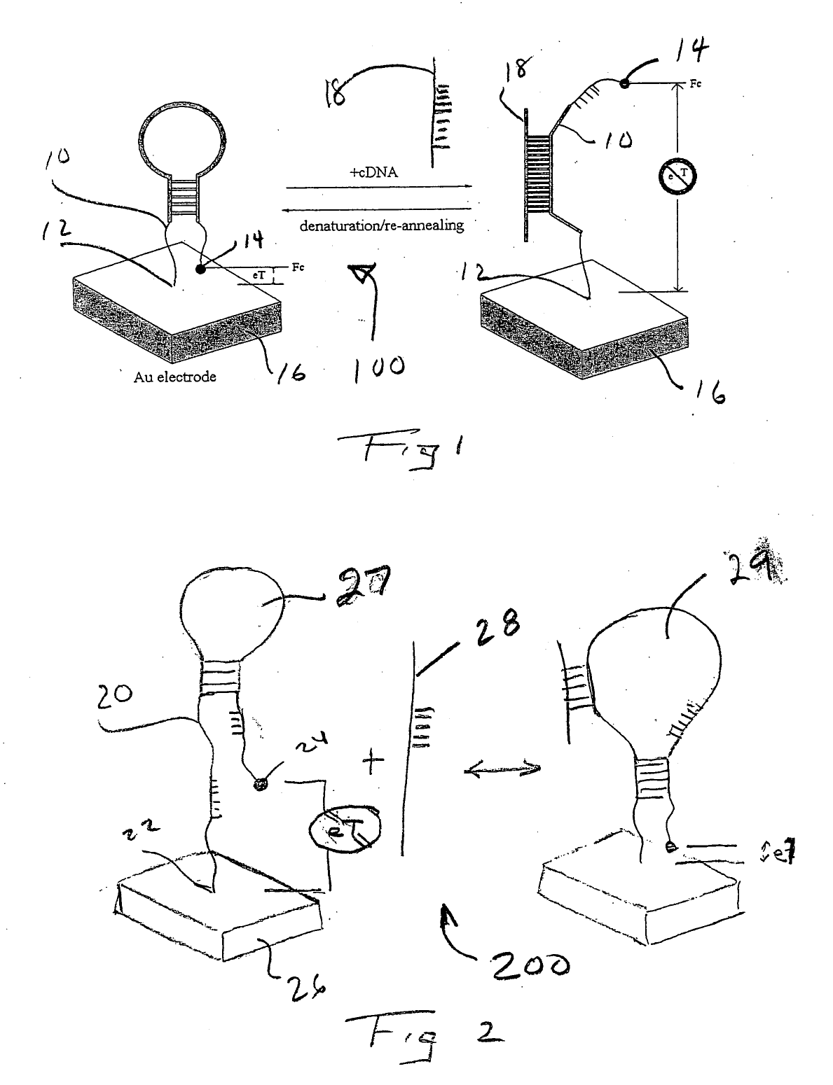

As shown in FIG. 1, the E-DNA sensors can employ a structured, stem-loop (hairpin-like) DNA with an electroactive label to detect hybridization events. Hairpin-like DNA is an extremely interesting structure that forms the basis of the fluorescent, “molecular beacon” approach for homogeneous, optical hybridization detection [Tyagi S and Kramer F. R., Nat Biotechnol 14, 303-308 (1996)]. In the DNA stem-loop, the base sequence has been designed such that the structure is initially in the folded “hairpin” configuration. The structure converts to the linear double helix form after hybridization with its specific complementary base sequence. The existence of the stem-loop structure in the design provides an on/off switch as well as a stringency test sufficient to discriminate single-base mismatches.

In sensor 100 of FIG. 1, a hairpin-like oligonucleotide 10 possessing for example a thiol 12 and a redoxable chemical moiety 14 such as for example, a ferrocene group or a methylene blue group, is immobilized on a gold electrode 16 via self-assembly. In the closed state, oligonucleotide 10 presents a stem-loop structure that localizes the redoxable chemical moiety 14 in close proximity to the gold surface 16. Thus the distance between the gold and redoxable chemical moiety is short enough for facile electron transduction (eT) thereby enabling redox of the redoxable chemical moiety in response to potentials applied via electrode 16. In the open state (after hybridization) with c DNA 18, electron transfer between the redoxable chemical moiety 14 and the electrode 16 is blocked since moiety 14 is separated from the electrode surface.

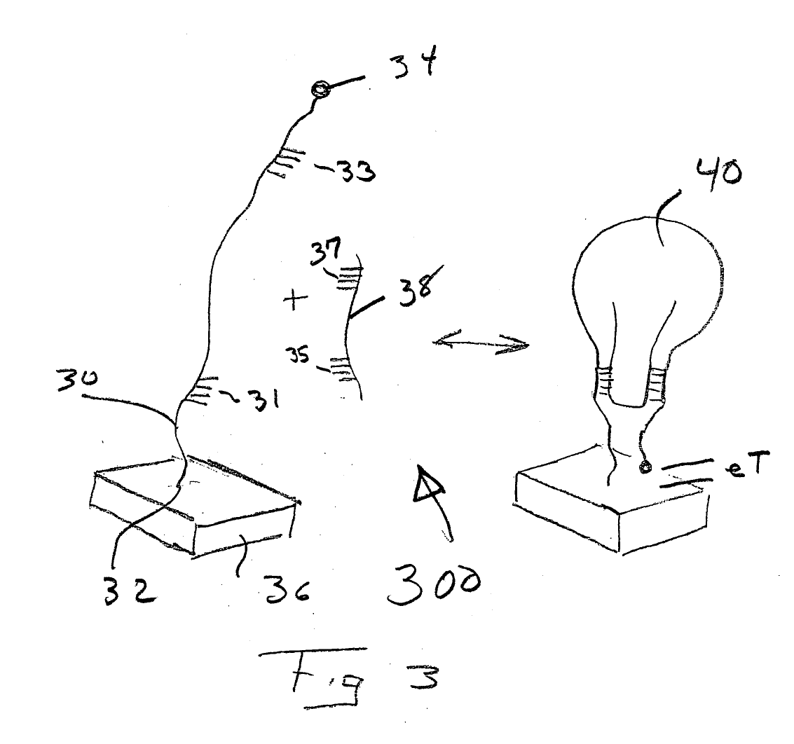

In the embodiment 100 described in FIG. 1, the E-DNA sensor suffers from being a “signal-off” sensor. That is, in response to its target, the electrochemical signal is abolished. This renders that embodiment of the E-DNA detector vulnerable to false positives arising via disruption of the stem-loop sensor element by environmental conditions or physical degradation (e.g. by nucleases). As shown in FIG. 2 with the appropriate oligonucleotide design “signal-on” E-DNA-type sensors 200 can be engineered, thus silencing false positives arising due to chemical or enzymatic destruction of the sensor element. The appropriate structure contains an appropriate DNA oligonucleotide probe 20 attached to or adjacent to electrode 26 at end 22. The other end of probe 20 carriers a redoxable moiety 24. In one configuration, probe 20 contains a moderate length hairpin 27 that positions the electroactive label 24 away from the electrode 26. That hairpin configuration 27 thermodynamically competes with a less stable hairpin configuration 29. The less stable hairpin 29 will, in contrast, position the label 24 in proximity to the electrode 26. Upon hybridization with target 28 the more stable hairpin 27 is disrupted, allowing the less stable hairpin 29 to form and bring the label 24 into signaling distance.

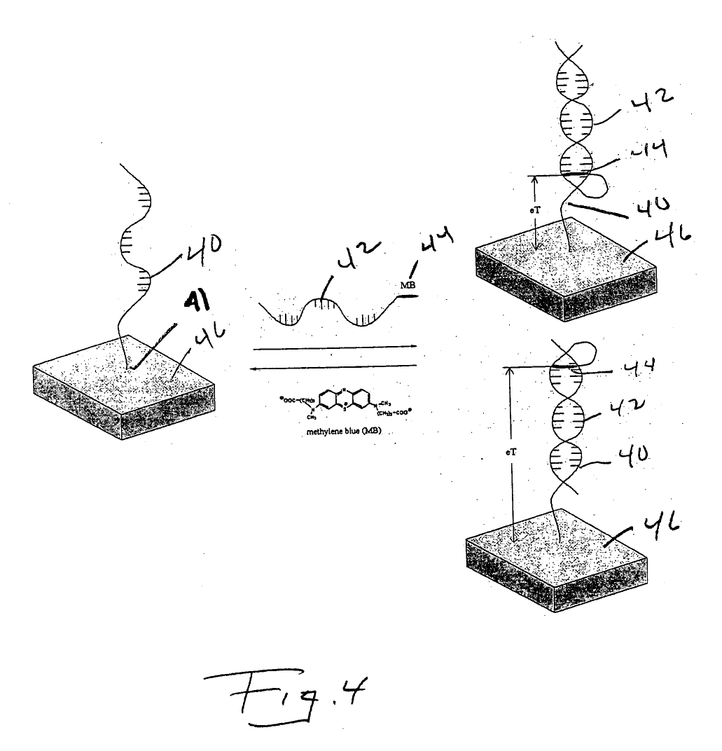

In another embodiment, as shown in FIG. 3, a DNA probe 30 may be coupled near or to electrode 36 via bond 32. The end of probe 30 distant from the point of attachment 32 is labeled with redoxable moiety 34. In the absence of target 38, probe 30 is “open” and label 34 is a long distance from electrode 36. Probe 30 contains regions 31 and 33 which are complementary to regions 35 and 37 on target 38. When target 38 and probe 30 are subjected to hybridization conditions, target 38 bridges across probe 30 to form loop 40 and thus positions redoxable moiety 34 near enough to electrode 36 that electron transduction can be induced and detected.

As shown in FIG. 4, one can also achieve a readable signal based on hybridization in systems not involving loop stem complexes. In FIG. 4, an oligonucleotide 40 possessing a terminal thiol group or other suitable binding group is immobilized at a gold electrode 46 via bond 41. A target 42 bearing redoxable label 44 is brought into proximity to the bound oligonucleotide 40. In the absence of target there is no signal. Upon hybridization with the target, the label is brought to a distance eT from the electrode and creates a electrochemical signal.

In this embodiment, the hybridization utilizes an electrochemical approach with a “signal-on” feature to identify DNA tags. The strategy demonstrated in FIG. 4 involves a gold electrode 46 and a DNA probe strand 40 without electroactive labels. The probe sequence is designed to be complementary to the DNA authentication tag 42 and contains a 5′ thiol. The probe is self assembled on the gold surface through gold-thiol chemistry. The tag 42 contains methylene blue as the electroactive label 44 at either its 5′ end or 3′ end. The tag may be encapsulated or otherwise secreted in documents or drugs. Prior to detection, the gold electrode has no signal since it has only the probe DNA. After hybridization such as with tag eluted from authenticated materials, the label is brought to the electrode surface and creates the electrochemical signal. The “signal-on” process is due to either the direct electron tunneling into the redox molecule from the gold electrode (upper image), or through electron transfer mediated by the DNA double helix (lower image) (Boon, E. M., Salas, J. E. & Barton, J. K., Nat. Biotechnol. 20, 282-286 (2002).). Since the signal is created only after hybridization, this approach offers the advantage of being insensitive to environmental contaminants.

These are but four representative configurations for the E-DNA sensor. Any probe configuration which will present different configurations in the presence and absence of target DNA, and which can reposition a redox label in electrically distinguishable different proximities to the sensing electrode can be used.

Representative Materials

In the embodiments just described the redoxable chemical moiety has been ferrocene or methylene blue. More generally, any redoxable chemical moiety that is stable within the electrochemical window of stability of DNA can be used. Other examples include, but are not limited to the following: viologen and redoxable species such as anthraquinone, ethidium bromide, daunomycin, and their derivatives.

In the preferred embodiment, the electrode is fabricated from known electrode materials such as, for example, gold, silver, platinum, carbon, or silicon. Gold gives good results.

In a preferred embodiment, the surface of the electrode is functionalized with the DNA probe structure through self-assembly such as through the well-established Au—S chemistry of self assembly.

It is also preferred when the electrode surface, functionalized with the DNA probe structure, is subsequently passivated by materials such as 2-mercaptoethanol, (2-ME), 6-mercaphohexanol or mercaptoalkanols generally (HS—(CH₂)ₙ—OH with n=2˜18) and the like.

When the electrochemical DNA sensor comprises a stable redoxable chemical moiety the sensor is readily reusable.

Generally, the stem-loop DNA structures are loosely packed on the Au surface in order to minimize steric effects that could interfere with hybridization.

Preferred embodiments for the stem-loop DNA structure are well known in the art. The stem-loop DNA structure is designed such that the five bases at its 5′-end and 3′-end are fully complimentary. The base sequence in the loop is chosen so as to be complementary to the specific base sequence to be detected in the target DNA.

It is often preferred to use a “stem” which is G-C rich in order to enhance its stability.

In some embodiments, the probe structure comprises an oligomer of neutral peptide nucleic acid (PNA) in place of the DNA oligonuceotide to allow hybridization to occur at ambient ionic strengths. In addition to silencing and detecting false positives, degradation of the sensor element can be avoided by building the stem-loop element from peptide nucleic acid (PNA). PNA is chemically and enzymatically robust and, because it is uncharged, forms stronger duplexes with DNA or RNA than ssDNA. Thus, there are clear advantages to “E-DNA” sensors comprising synthesized PNA sensor elements.

The DNA probe may be attached to the electrode via a “molecular-wire” such as, for example, an oligo(phenylene vinylene) in order to facilitate electron transfer.

The sensor can also employ optemers. An aspect of the E-DNA detection is the electrochemical detection of a target-induced conformational change. This means that this invention may be generalizable to other types of tags and analytes where conformational change occurs upon binding, such as protein folding or optemer folding based biosensors.

Optemers are DNA or RNA molecules that adopt well-defined tertiary structures analogous to natural enzymes. Optemers have emerged as promising therapeutic and diagnostic tools [Chang, K. Y. & Varani, G., Nature Struct. Biol. 4, 854-858 (1997); Burgstaller, P., Girod, A. & Blind, M., Drug Discov. Today 7, 1221-1228 (2002); Wilson, D. S. & Szostak, J. W., Annu. Rev. Biochem. 68, 611-647 (1999)]. Well developed in-vitro selection has been able to produce optemers for virtually any given target [Wilson, D. S. & Szostak, J. W., Annu. Rev. Biochem. 68, 611-647 (1999); Griffiths, A. D. & Tawfik, D. S., Curr. Opin. Biotech. 11, 338-353 (2000).]. Given these advantages, oligonucleotide optemers are anticipated to play an important role in next-generation biosensing elements [Sullivan, C. K. O., Anal. Bioanal. Chem. 372, 44-48 (2002); Robertson, M. P. & Ellington, A., Nature Biotech. 17, 62-66 (1999)].

DNA or RNA optemers that undergo significant conformational changes upon binding specific analytes are readily available. In vitro selection techniques are able to isolate highly affinitive RNA or DNA optemers that bind almost any arbitrary small molecule, biomacromolecule or cell type. Many optemers undergo significant conformational changes upon analyte binding. Alternatively, although insignificant signal changes are expected for those optemers that undergo subtle conformational changes, it is feasible to accomplish the analyte detection via combining a recently proposed optemer self-assembly approach [Stojanovic, M. N., de Prada, P. & Landry, D. W., J. Am. Chem. Soc. 122, 11547-11548 (2000)] For example, optemers rationally dissected into two halves, with one immobilized at electrode surfaces while the other tagged with electroactive label, are expected to be split in the absence of analytes while self-assembled upon analyte binding. Thus the approach described here can be generalized from stem-loop structures to DNA and RNA optemers and thereby to sensing platforms directed against essentially any water soluble analyte.

Reaction Conditions and Detection Methods

The hybridization events which are sensed by the detectors and methods of this invention are carried out in aqueous liquid environment. These aqueous environments are preferably but optionally rendered at least somewhat ionic by the presence of dissolved salt. It is generally understood that hybridization reactions are more facile in ionic environments and this holds true in the present setting. “Salt” is defined to include sodium chloride but also any other water-soluble alkaline earth or alkyl metal ionic materials. While there may be advantages to particular salt materials or levels, they are not seen to be critical to the practice of this invention. Representative salt levels can be as high as about 4 or 5 molar, in some cases and as low as nearly zero. In the examples, 1 molar NaCl is generally used. Thus, salt levels of from about 0.05 to about 2 molar are presently preferred.

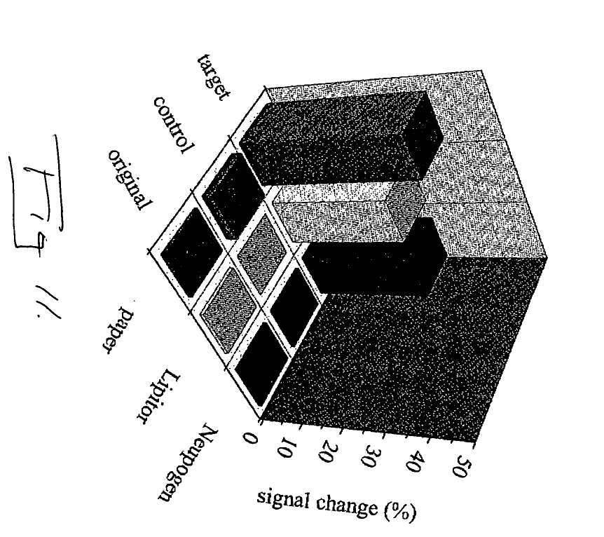

The hybridization can be carried out in the presence of agents and additives that promote the desired hybridization or diminish background nonspecific interactions. For example, one can add up to 10% by weight or volume (based on the amount of aqueous environment) and particularly from about 1 or 2% to about 10% of one or more polyols. Representative polyols include glycerol, ethylene glycol propylene glycol sugars such as sucrose or glucose, and the like. One can also add similar levels of water soluble or water dispersible polymers such as poly(ethylene glycol) or poly(vinyl alcohol) or the like. A third representative additive is up to about 1 or 2% by weight (again based on the liquid substrate) of one or more surfactants such as triton X-100 or sodium dodecyl sulfate. All of these agents are electrochemically silent at the potentials observed with the sensors and methods of the invention. As a comparison of the results shown in FIG. 11 with the results set forth is FIG. 10 make clear, additive addition can lead to dramatic improvements.

Hybridization can be carried out at ambient temperature, although any temperature in the range of from about 20 to about 40 or 45 C. can be used

Hybridization times should be a short as possible for convenience. Times as short as a few minutes (say 2 to 5 minutes) can be used up though an hour or so. We have had good results with hybridization times of from about 15 to about 45 minutes.

Multiplexing

False positives can be identified via multiplexing—using multiple, electrochemically distinct labels—such that the sensor and one or more control elements are integrated into a single sensor pixel. By employing multiple labels with narrow, non-overlapping redox potentials, 2-5 or possible more distinct sequences can be simultaneously interrogated on a single electrode. This enables the inclusion of internal controls—elements that are not complementary to known sequences that would respond to false positives arising due to non-specific disruption or degradation of the stem-loop. Multiplexing will also facilitate signal redundancy, alleviating the risk of masking in the unlikely event of contaminants with redox potentials precisely where the primary label reports. In addition to exhibiting narrow, non-overlapping redox peaks, the appropriate labels for multiplexing should be stable and synthetically facile. Electroactive labels that meet these requirements, include a large number of ferrocene [Brazill, S. A., Kim, P. H. & Kuhr, W. G., Anal. Chem. 73, 4882-4890 (2001)] and viologen derivatives (Fan, C., Hirasa, T., Plaxco, K. W. and Heeger, A. J. (2003) Langmuir, and any redoxable species, such as methylene blue, anthraquinone, ethidium bromide, daunomycin.

Improved sensitivity: AC voltammetric methods are commonly employed in an effort to delineate between redox and charging currents based on the different timescales for the two processes; double layer formation is limited only by ion mobility and thus equilibrates rapidly, whereas redox currents are limited by Marcus-type barriers and is orders of magnitude slower. Sinusoidal voltammetry (SV) or pulsed voltammetry has proven particularly useful; in addition to the SV frequency spectrum, time course data is obtained at each harmonic frequency element by performing the digital equivalent of a lock-in amplifier (Brazill S A, Bender S E, Hebert N E, et al. J. Electroanal. Chem., 531, 119-132 (2002)). That is, the instantaneous current is monitored at the optimum phase angle for the signal of interest, thus greatly increasing the sensitivity and selectivity over traditional voltammetric techniques. This temporal deconvolution enables a large increase in peak to charging current ratios and thus an improvement in the E-DNA sensitivity by orders of magnitude. Cyclic voltammetry is also used.

Improved peak currents: The use of multiple electroactive reporters will significantly increase the sensitivity. A straightforward approach to this end would be to label the single sensor strand with multiple electroactive reporters. The sensor DNA element in FIG. 1 is modified on the 2′ position of the terminal nucleotide, but modification of internal nucleotides is equally facile and should not significantly reduce the stability of the stem element. Because the electroactive label is isolated from the nucleotide by a pentyl linker, the labels will not interact with one another and thus multi-labeled sensor elements will exhibit redox peaks at the same potential (and peak width) as single-labeled construct. Because peak current is proportional to the number of electron acceptors/donors this approach will only improve peak currents by a factor of 2-5, with the upper limit corresponding to the number of electron acceptors that can be packed on the 5 bases in the terminal stem sequence.

Electrocatalysis, in contrast, provides a potential means of increasing peak currents by orders of magnitude. The approach works by the addition of an electrochemical mediator, such as ferrocyanide, that is not reduced by the electrode but can be reduced by the ferrocene label (Boon, E. M., Ceres, D. M., Drummond, T. G., Hill, M. G., Barton, J. K. (2000) Nat. Biotech., 18, 1096-1100). Thus, in the presence of ferrocyanide, the electrode repeatedly reduces each ferrocene, thus catalytically increasing peak currents. This approach leads to a sensor that is no longer reagentless.

Tag Detection and Authentication

This invention provides a reagentless, electronic means of rapidly, specifically and inexpensively detecting DNA-based authentication tags optionally in the presence of security-relevant levels of masking DNA. The method is suitabile for the authentication of a wide range of items ranging from documents to both orally ingested (solid) and injectable (liquid) drugs.

With this E-DNA sensor and optionally alternating current voltammetry (ACV), it is possible to read out the DNA information whether it be on packaging or on a label or the like or deposited on or dispersed in a solid or liquid (e.g. oral or injectable drugs). Only a small amount of DNA oligos (5 ng for the paper and 20 ng for the drugs) are necessary as the authentication tag. More importantly, the E-DNA sensor can discriminate against a great excess of non-cognate DNA, which acts as masking DNA in order to thwart efforts to forge the authentication tag via cloning or sequencing.

Therefore, DNA oligomers can act as authentication tags and the E-DNA sensor conveniently identifies the hidden DNA sequence information in minutes. Given the simplicity and usefulness of this novel technology, it finds application in a variety of markets.

A DNA oligomer that takes part in the hybridization to a probe sequence is employed as an authentication tag. A droplet of such a DNA oligomer is mixed with a multi-fold concentration such as 50 fold to 500,000 fold, e.g. 10,000 fold of non-cognate, masking DNA, and used in document and drug authentication and the like.

In this application, the DNA solution containing both DNA authentication tag and masking DNA may be deposited on a piece of filter paper or similar inert material. The paper is dried in the air and associated with (attached or the like) to the object to be authenticated. In the authentication stage, the paper is immersed in salt water to elute the tag. The eluted tag is ready for E-DNA detection.

In similar embodiments, DNA tags may be admixed in a solid, for example, Lipitor powder and thereafter dispersed in salt water and tested by the E-DNA sensor.

The E-DNA authentication strategy is particularly robust to counterfeiting. The extremely high selectivity of the E-DNA sensor enables us to specifically detect the authentication DNA sequence even in the presence of a 10,000-fold excess of non-cognate “masking” DNA. This high level of masking would render it extremely difficult to forge the authentication tag via the polymerase chain reaction (PCR), cloning or other amplification and/or copying methods. Moreover, the E-DNA approach is presumably also suitable for the detection of peptide nucleic acid (PNA)-based authentication tags. Because PNA cannot be amplified or sequenced via enzymatic methods the use of such tags would render the approach still more robust to copying-based counterfeiting. In contrast, as described, the E-DNA approach could potentially be partially circumvented via dilution- the extraction and diluting the authentication tag from one document and its application to several forged documents-or via the inclusion of materials (denaturants, nucleases, etc.) that would disrupt or overwhelm stem hybridization. Attacks based on the former, however, can be frustrated via measurements of the absolute DNA concentration in the authentication sample and ratiometric measurements of the absolute DNA content versus the concentration of authentication tag. Similarly the latter circumvention can be thwarted by ratiometric measurements of authentication tag versus control sequences known to be absent in authentic goods. Given that the small electrode size and reagentless nature of the E-DNA sensor renders it particularly well suited for dense, electronic sensor arrays such ratiometric measurements are not a significant hurdle.

Microelectrodes and arrays: Because E-DNA is an electronic sensor, advances in electrophoretically-improved hybridization times can be applied [Cheng, J., Shoffner, M. A., Hvichia, G. E., Kricka, L. J., Wilding, P. (1996). Nuc. Acid Res., 22, 380-385; Cheng, J., Sheldon, E. L., Wu. L., Uribe, A., Gerrue L. O, Heller, M. O'Connell, J. (1998). Nat. Biotech. 16, 541-546]. Moreover, because of its direct integration into electronics and excellent scalability (in the Example 1, 2 mm² electrodes were used, but E-DNA's impressive signal strength suggests that significantly smaller electrodes can be employed), E-DNA is well suited for applications in electronic gene detection arrays. To this end, biomaterials can be deposited onto specific pixels of gold “nanode” arrays and electrochemically addressed.

In a more preferred embodiment, the microelectrodes are arrayed in the format of N “pixels” with each pixel containing a unique stem-loop or the like DNA structure and with all microelectrodes electrochemically addressable, thereby enabling detection of N different targets.

As demonstrated in the Examples, the bioelectronic DNA sensor described herein is both sensitive and highly selective. The sensitivity and selectivity of the E-DNA sensor is better than that of typical CCD-based fluorescent detectors, and is comparable to a recently proposed, conjugated polymer-based fluorescence amplification method [Gaylord, B. S., Heeger, A. J. & Bazan, G. C., Proc. Nat. Acad. Sci. USA 99, 10954 (2002); Moon, J. H., Deans, R., Krueger, E. & Hancock, L. F., Chem. Commun., 104-105 (2003)]. The key sensing element, the electroactive stem-loop, is compatible with normal solid-state synthesis of oligonucleotides. Moreover, the surface assembly process is robust and facile. Since the entire set-up can be conveniently prepared and generalized to be consistent with chip-based technology, the novel, reagentless detection described here provides a promising alternative to fluorescence-based sensors.

The following general methods and specific examples are presented to illustrate the invention and are not to be considered as limitations thereon.

### EXAMPLES

### Example 1

### Fabrication of the Stem-Loop DNA Structure

Ferrocene carboxylic acid was purchased from Aldrich (Milwaukee, Wis.), 1-Ethyl-3-(3-dimethylaminopropyl)-carbodiimide (EDC) and N-hydrosuccinimide ester (NHS) were obtained from Sigma (Milwaukee, Wis.). Ferrocene succinimide ester (Fc-NHS) was prepared as described in the literature [Takenaka, S., Uto, Y., Kondo, H., Ihara, T. & Takagi, M. Anal. Biochem. 218, 436. (1994)] and confirmed by ¹H NMR. Oligonucleotides were obtained from Synthegen (Houston Tex.). The sensor oligonucleotide, sequence 5′-NH₂—(CH₂)₆-GCGAG GTA AAA CGA CGG CCA GT CTCGC-(CH₂)₆—SH-3′ (oligo 1), contained a 5′ hexamethylene amine and a 3′hexamethylene thiol group . Fc-NHS was dissolved in a small volume of dimethyl sulfoxide and then diluted in a 0.1 M Na₂CO₃ buffer (pH 8.5) containing 0.1 mM of oligo 1. This mixture was stirred overnight at room temperature. The final product (oligo 1-Fc) was purified by HPLC on a C18 column and confirmed by electrospray mass spectroscopy. The sequences of the target and control DNA oligos were 5′-ttttt ACT GGC CGT CGT TTT AC tcttt-3′ and 5′-CGT ATC ATT GGA CTG GCC ATT TAT-3′. All solutions were prepared with nano-pure water.

### Example 2

### Preparation of the Functionalized Au Electrode

[ ] Polycrystalline Au disks (1.6 mm diameter) (BAS Inc., West Lafayette, Ind.) were used as working electrodes. The protocol for gold electrode preparation has been previously described [Fan, C., Gillespie, B., Wang, G., Heeger, A. J. & Plaxco, K. W., J. Phys. Chem. (B) 106, 11375-11383 (2002)]. The cleaned Au electrode was rinsed, dried under argon and then immediately incubated overnight in 1 M oligo 1-Fc, 10 mM phosphate buffer with 0.1 M NaCl, pH 7.4. Prior to use, the oligo 1-Fc was pre-treated with tris-(2-carboxyethyl)phosphine to break the disulfide bond and then purified by spin column. The modified electrode was washed with water, dried under argon and incubated in 1 M NaClO₄ solution prior to use.

The gold surface was then functionalized by oligo 1 (see Example 1) through the well-established Au-S chemistry of self-assembly. Previous studies have demonstrated that this self-assembly process is only feasible in the presence of salt; high ionic strength leads to high surface density and closely packed DNA strands while low ionic strength produces loosely packed DNA strands [Boon, E. M., Salas, J. E. & Barton, J. K., Nature Biotech. 20, 282-286 (2002)]. For this Example, a relatively low ionic strength (0.1 M NaCl) was chosen to make a loosely packed surface in order to minimize steric effects that could interfere with reversible hairpin formation (see FIG. 1). The prepared surface was subsequently passivated by 2-mercaptoethanol (2-ME). This process has been reported to “cure” the relatively disordered self-assembled monolayer (SAM) by gradually displacing nonspecifically adsorbed oligonucleotides [Herne, T. M. & Tarlov, M. J., J. Am. Chem. Soc. 119, 8916-8920 (1997)]. This oligonucleotide-containing, passivated surface has proven to be resistant to random DNA sequences, as reported previously [Herne, T. M. & Tarlov, M. J., J. Am. Chem. Soc. 119, 8916-8920 (1997)] and independently confirmed in our labs by monitoring with a quartz crystal microbalance.

### Example 3

### Characterization of the E-DNA Modified Electrode

The stem-loop structure localizes the ferrocene tag in close proximity to the gold surface (see Example 2 and FIG. 1) and thereby ensures that the distance between the gold electrode and the ferrocene moiety is short enough for facile electron communication.

Cyclic Voltammetry (CV) was performed using a CHI 603 workstation (CH Instruments) combined with a BAS C-3 stand. A platinum electrode was used as a pseudo-reference electrode while potentials are reported versus the normal hydrogen electrode (NHE). Background subtraction was conducted in some cases using Origin 6.0 (Microcal Software, Inc.) in order to remove non-Faradayic currents and improve signal clarity [Fan, C., Gillespie, B., Wang, G., Heeger, A. J. & Plaxco, K. W., J. Phys. Chem. (B) 106, 11375-11383 (2002).Hirst, J. et al. J. Am. Chem. Soc. 120, 7085-7094 (1998). Bard, A. J. & Faulkner, L. R. Electrochemical Methods (John W. Willey & Sons, New York, 2001)]. All experiments were conducted at room temperature.

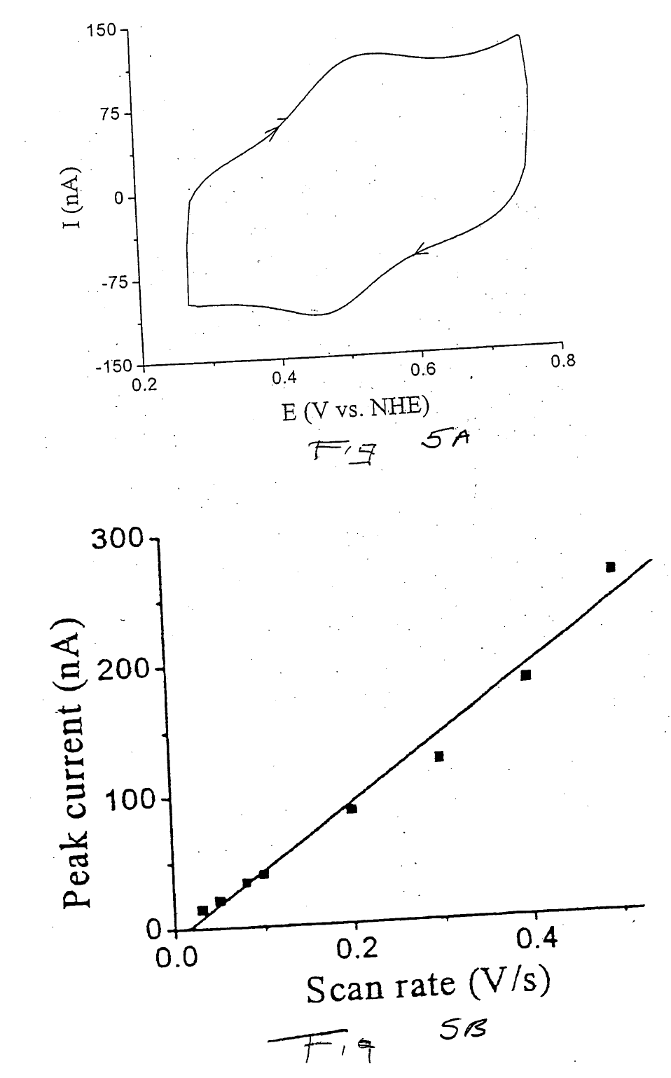

In the absence of target DNA, ferrocene redox peaks were observed (FIG. 5a). For comparison, a bare gold electrode or gold modified with either 2-ME or 2-ME/mercapto-oligonucleotides lacking ferrocene produces featureless CV curves in the same potential window. The apparent formal potential of the electroactive label is E⁰=0.492 V, as estimated from E_{1/2}=(E_{red}+Eₒₓ)/2. This value falls within the typical redox potential range of ferrocene (E⁰ of ferrocene is slightly sensitive to the environment, but remains within a relatively limited potential range) [Brazill, S. A., Kim, P. H. & Kuhr, W. G., Anal. Chem. 73, 4882-4890 (2001)]. Therefore, this peak pair was ascribed to the redox conversion of ferrocene labels in close proximity to the gold electrode. It is known that high salt concentration is required for the formation of short stem-loop structures as a result of the electrostatic repulsion between negatively charged DNA chains [Herne, T. M. & Tarlov, M. J., J. Am. Chem. Soc. 119, 8916-8920 (1997)]. We found that some freshly modified electrodes do not produce redox peaks without prior incubation in 1 M NaClO₄. This result provided strong evidence that the formation of the stem-loop structure facilitated the electron transfer between the gold electrode and ferrocene by constraining the ferrocene label in close proximity to the electrode surface. This result also implied that the use of neutral peptide nucleic acids (PNA) in place of the DNA might provide significant advantages by allowing hybridization to occur at ambient ionic strengths.

Modulating the scan rate of the CVs provided further evidence that ferrocene was confined at the electrode surface by the formation of the stem-loop structure. Peak currents of the ferrocene redox reaction (Iₚ)were directly proportional to scan rates (FIG. 5b), consistent with a surface-confined electrochemical reaction (in contrast to Iₚ being proportional to the square-root of the scan rate characteristic of diffusion-controlled electrochemical reactions) [Bard, A. J. & Faulkner, L. R. Electrochemical Methods (John W. Willey & Sons, New York, 2001)]

### Example 4

### Target DNA Detection

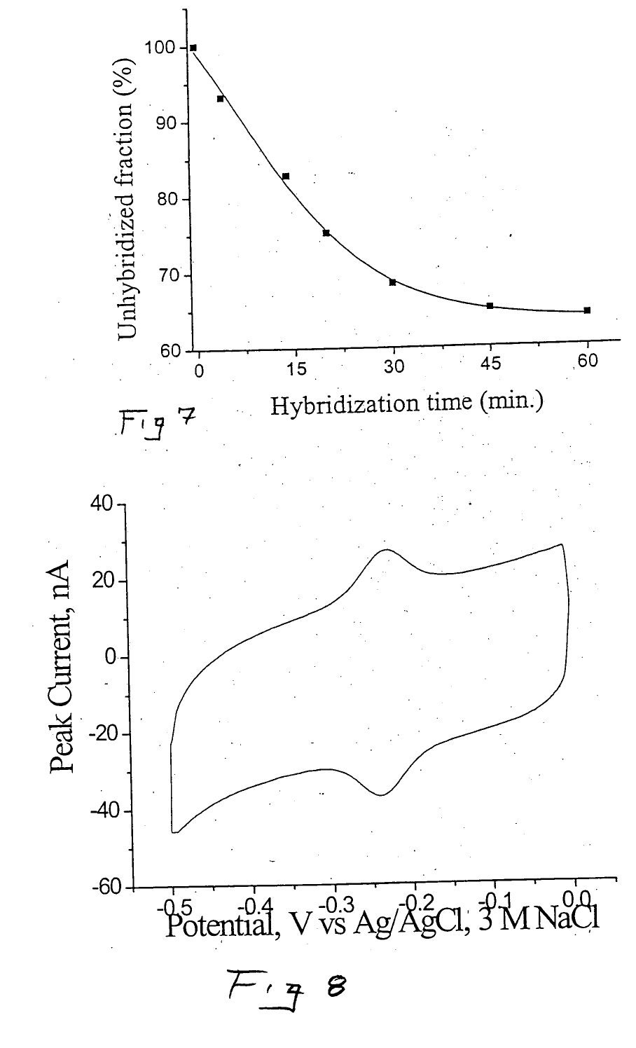

When the stem-loop structure meets a sequence complementary to the loop region (17 bases), hybridization breaks the less stable stem structure and isolates the ferrocene from the electrode surface. Thus, incubating a stem loop-modified electrode in a 5 M cDNA (oligo 2, see Example 1) solution containing 1 M NaClO₄ eliminated the ferrocene reduction and oxidation peaks within ˜30 min (FIG. 5a). After incubating the electrode with 500 pM cDNA solution and monitoring the hybridization process electrochemically, we observed that the electrochemical signal attenuates with a time constant of approximately 30 min (FIG. 7).

### Example 5

### Sensor Sensitivity

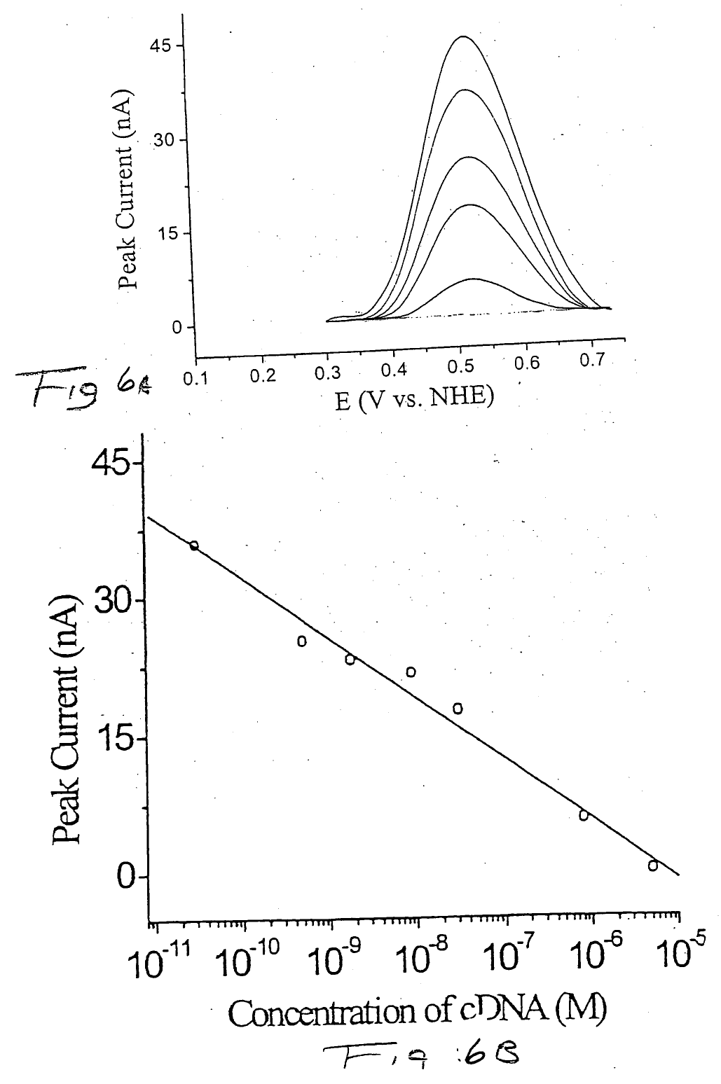

Employing a fixed 30-minute incubation time, the sensitivity of the sensor was tested. We observed easily measurable decreases in peak intensity at target DNA concentrations as low as 10 pM (FIG. 6). Peak currents are logarithmically related to target concentration across the almost six decade range of sample concentrations we have investigated.

### Example 6

### Sensor Selectivity

The E-DNA sensor is highly selective. Employing a fixed 30-minute incubation time, we have tested the sensitivity of the sensor. We observe easily measurable decreases in peak intensity at target DNA concentrations as low as 10 pM (FIG. 6a). Peak currents are logarithmically related to target concentration across the almost five decade range of sample concentrations we have investigated (FIG. 6b).

No significant signal change is observed for electrodes incubated in DNA-free hybridization buffer or in the presence of the highest non-target DNA concentrations we have investigated (10 M oligo 3, see Example 1). Thus the selectivity of the sensor relative to a random target sequence is in excess of 10⁶.

### Example 7

### Sensor Regeneration

The electrochemical DNA sensor is readily reusable. Washing the electrode with 1 M NaClO₄ at 95 C. and re-challenging with the target sequence, we have successfully recovered up to ˜80% of the original signal. The minor loss of the signal during recovery presumably arises due to the relative instability of ferrocene at high temperature.

### Example 8

### Fabrication of the Stem-Loop DNA Structure with MB Label

Oligonucleotides were obtained from Synthegen (Houston, Tex.). The sensor oligonucleotide, 5′-HS—(CH2)6-GCGAGGT AAAACG ACGGCC AGTCTCGC-(CH2)6-NH2-3′ (oligo 1), contains a 5′ hexamethylene thiol and a 3′ hexamethylene amine. A methylene blue (MB) tag was conjugated to oligo 1 through coupling the succinimide ester of MB (MB-NHS, EMP Biotech, Germany) with the 5′ amine of oligo 1. The final product (oligo 1-MB) was purified by HPLC on a C18 column and confirmed by electrospray mass spectroscopy. The sequences of the target and control DNA oligos were 5′-ACTGGCCGTCGTTTTAC-3′ (oligo 2) and 5′-CGTATCATTGGACTGGC-3′ (oligo 3) respectively. The oligo 2 is fully complementary to the loop sequence while the control oligo 3 is a random sequence unrelated to the probe sequence which is used as the masking DNA.

### Example 9

### Preparation of the Functionalized Au Electrode

Polycrystalline Au disks (1.6 mm diameter) (BAS Inc., West Lafayette, Ind.) were used as working electrodes. The E-DNA sensor was constructed by assembling the MB-labeled DNA stem-loop at the gold electrode. In order to construct the sensor as demonstrated in FIG. 4, a 0.1 mM solution of the stem-loop oligo 1-MB (with 100 mM NaCl, 5 mM MgCl2 and 10 mM phosphate buffer at pH 7.0) was self-assembled on an extensively cleaned gold surface (Leopold, M. C., Black, J. A. & Bowden, E. F., Langmuir 18, 978-980 (2002); Fan, C., Gillespie, B., Wang, G., Heeger, A. J. & Plaxco, K. W., J. Phys. Chem. (B) 106, 11375-11383 (2002).). The prepared surface was subsequently passivated with excess 6-mercaptohexanol at 1 mM for ˜2 hrs. The modified electrode was thoroughly rinsed, dried and then incubated in 1 M NaCl prior to use.

### Example 10

### Description of the MB Labeled E-DNA Sensor

Cyclic voltammetry (CV) and AC voltammetry (ACV) were performed at room temperature using a CHI 603 workstation (CH Instruments, Austin, Tex.). In ACV, we employ 10 Hz frequency and 25 mV amplitude. Potentials are reported versus the Ag/AgCl, 3 M NaCl reference electrode (BAS Inc.). A platinum wire was used as the counter electrode.

MB, as well as the previously employed ferrocene, is readily redoxable at gold electrodes. As demonstrated in FIG. 8, a pair of well-defined peaks were obtained for E-DNA in the absence of targets, which corresponds to the redox conversion of the MB label in close proximity to the gold electrode. Upon hybridization with complementary sequence to the loop range, the unfolding of the stem-loop moves the MB away from the electrode surface, which significantly decreases the electrochemical signal.

FIG. 8 provides a cyclic voltammogram for a gold electrode modified with the MB tagged, stem-loop oligonucleotide in the absence of target DNA (scan rate of 0.1 V/s). The electrolyte is 10 mM phosphate buffer/1 M NaCl, pH 7.0.

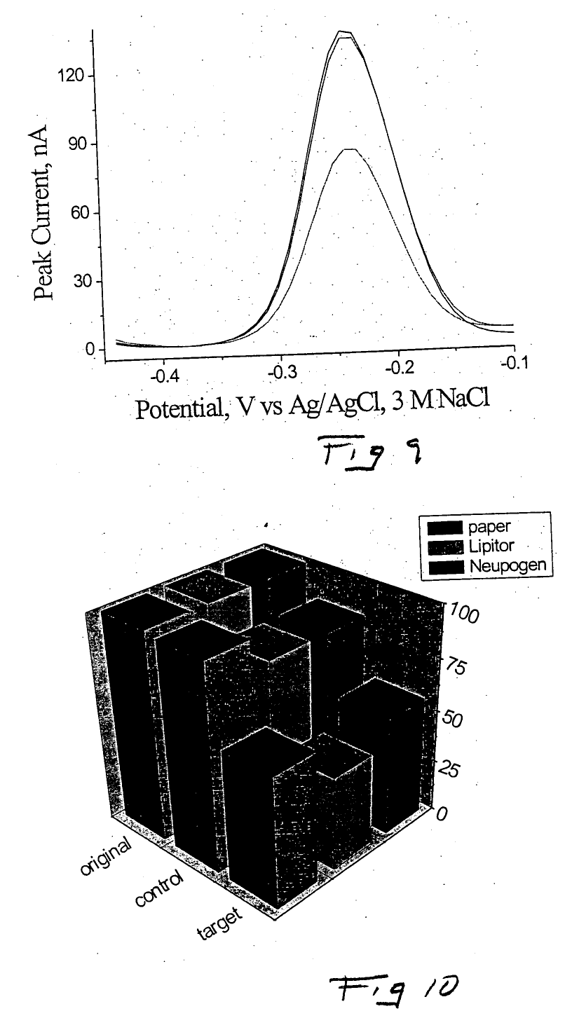

The MB-labeled E-DNA sensor works in alternating current voltammetry mode (ACV). ACV typically involves the application of a sinusodially oscillating voltage to an electrochemical cell which has proven to effectively reduce charging (background) current (O'Connor, S. D., Olsen, G. T. & Creager, S. E. J. Electroanal. Chem. 466, 197-202 (1999).). As shown in FIG. 9, the ACV of E-DNA has a nearly flat background, making the comparison between curves both convenient and quantitative. Consequently, ACV was used in the following DNA authentication studies.

FIG. 9 provides AC voltammograms for the E-DNA sensor before the test, and after the test with DNA microdots containing masking DNA (50 mg) only, and masking DNA (50 mg) mixed with target DNA (5 ng). The hybridization time was 30 minutes.

The employment of the MB label brings about three advantages. First, in the ferrocene labeled E-DNA sensor, the electrochemical experiments are best performed only in certain salt solutions (e.g., perchlorate), because ferrocene, if oxidized, is vulnerable to strong nucleophiles (e.g. chlorides) (Han, S. W., Seo, H., Chung, Y. K. & Kim, K., Langmuir 16, 9493-9500 (2000).). This limitation has been overcome via the employment of MB label, which is more stable in chloride solutions. Therefore, the use of MB labels not only frees the risk of using potentially dangerous perchlorates, but avoids the necessity of removing possible chloride contaminations.

Second, ferrocene has little affinity to DNA strands, therefore the labeled ferrocene dangles under the stem-loop which may increase the surface heterogeneity. This effect is reflected by the non-ideal electrochemistry of ferrocene, such as decreased electron transfer rates and broadened peaks, due to dispersion of kinetic and thermodynamic parameters (rate constants, formal potentials etc.) (Saccucci, T. M. & Rusling, J. F. J. Phys. Chem. (B) 105, 6142-6147 (2001); Clark, R. A. & Bowden, E. F. Langmuir 13, 559-565 (1997).). In contrast, MB, as a DNA intercalator, inserts into the stem double helix (Muller, W. & Crothers, D. M., Eur. J. Biochem. 54, 267-277 (1975); Boon, E. M., Salas, J. E. & Barton, J. K., Nat. Biotechnol. 20, 282-286 (2002).). This intercalation limits the diffusion of the label, which leads to much improved electrochemical behavior, including sharper peaks (less thermodynamic dispersion) and smaller peak separations (less kinetic dispersion) (FIG. 1). For example, for CVs at 100 mV/s, the n×FWHM (full width at half-maximum) has been reduced from ˜170 mV to ˜140 mV, and the n×DE has been reduced from ˜60 mV to ˜30 mV in the MB labeled E-DNA (n stands for the electron transfer numbers).

Third, MB is very stable against thermal degradation water and this will provide a more readily reusable sensor. This means that an MB-based sensor can be washed with hot water to remove hybridized target and give a good strong signal when reused.

### Example 11

### Document Authentication with E-DNA Sensor

The feasibility of encapsulating DNA sequence information in a piece of filter paper was tested. The E-DNA sensor was used as a convenient readout device. 1 ml of the DNA solution (˜5 ng oligo 2 with 10,000-fold excess of non-cognate DNA oligo 3) was added over a small cycle (˜3 mm diameter) printed on filter paper with a ball pen. Interestingly, the DNA solution was confined in this cycle, possibly due to the fact that the diffusion of the solution in the filter paper was hindered by the hydrophobic pen ink. This DNA microdot, after being dried, was cut from the paper and immersed in 20 ml salt water containing 10 mM phosphate buffer with pH 7.0 and 1 M NaCl for approximately 10 min. 2 ml of the eluted solution was placed at the E-DNA electrode surface. After 30-min hybridization, the ACV signal dropped by approx. 40%. As a control, the E-DNA signal remain and almost unchanged for a DNA microdot with only 50 mg masking DNA (oligo 3) (FIG. 9 and FIG. 10).

FIG. 10 provides comparisons among the E-DNA signals before and after counterfeiting test in filter paper, Lipitor and Neupogen.

This experiment clearly demonstrates that one needs only a very small amount of DNA oligo (˜5 ng) with designed sequence to “authenticate” the provenance of documents. This sequence information can be read through an E-DNA sensor with the appropriate probe DNA. The extremely high specificity has enabled one to mask the sequence information in 10,000-fold excess of non-cognate “masking” DNA. The high specificity in 10,000-fold excess of non-cognate “masking” DNA implies that it is nearly impossible to amplify the DNA authentication tag through polymerase chain reactions (PCR) and sequencing. Although this preliminary experiment was performed with filter paper, previous studies have proven it possible to encapsulate DNA in other substrates such as typical letter paper, with stability over two years at room temperature, where they nevertheless used time-consuming gel electrophoresis to read the DNA information (Cook, L. J. & Cox, J. P. L. Biotechnol. Lett. 25, 89-94 (2003).). Given the complexity of DNA sequence information (a 17-mer corresponds to ˜seventeen billion combinations), convenience of encapsulation, and readout of the described technology, this DNA authentication technology is promising for authentication of important documents.

### Example 12

### Thwarting Drug Counterfeiting with E-DNA Sensor

Lipitor tablets were selected as an example of orally ingested drugs and Neupogen as an example of injectable drugs. Lipitor is a cholesterol lowering drug (Warner-Lambert Export, Ltd), while Neupogen (Amgen) is a cancer-control drug that fights against Neutropenia, a disease with low white blood cell count.

The Lipitor tablets were ground into powder and a droplet (1 ml) of DNA (20 ng oligo 2 with 200 mg masking DNA) on the powder. After drying in the air, the powder was dispersed in 50 ml salt water followed by filtering to obtain the supernatant. For the Neupogen liquid, 1 ml Neupogen was mixed with 1 ml DNA (20 ng oligo 2 with 200 mg masking DNA), and then diluted into a 50 ml solution; 2 ml of this solution was pipetted on the gold electrode surfaces. The control experiments were performed in the absence of target DNA tag using only the masking DNA. As demonstrated in FIG. 10, we observed a significant decrease in the ACV signal after 30-min hybridization. In both cases, significantly smaller decreases of the corresponding signals were observed in the control experiments. The decreases in the control experiments possibly arise from the non-specific adsorption of some components of the drugs. It will be appreciated that one might wish tocontrol the reaction time and the concentration of target DNA in order to obtain optimized results in real sample detection. Nevertheless, due to the significant differences in response between the target DNA-containing experiments and the control experiments, it is possible to use the E-DNA sensor to read out the DNA information hidden in drugs.

### Example 13

### Reducing Background Signals with Additive Addition

The experiments set forth in Example 12 were repeated with one change. Glycerol (5% by volume) was present in the solutions pipetted onto the gold electrodes. The glycerol additive greatly reduced the background signal in the control samples and gave the change in signal shown in FIG. 11. This illustrates that by the addition of materials which block nonspecific interactions between masking DNA and the probe a much clearer and specific result is achieved.

What is claimed:

1. A detector for determining the presence of a target polynucleotide having a target nucleotide sequence comprising an electrode capable of sensing redox events in a redox moiety and an immobilized polynucleotide probe, the probe carrying a redox moiety and having a probe nucleotide sequence which hybridizes with the target nucleotide sequence and having a first configuration, in the absence of hybridization with the target polynucleotide, which locates the redox moiety in a first position relative to the electrode and having a second configuration, in the presence of hybridization with the target polynucleotide, which locates the redox moiety in a second position relative to the electrode, said first and second positions giving rise to distinguishable redox events detectable by the electrode.

2. The detector of claim 1 wherein the first position is closer to the electrode than the second position.

3. The detector of claim 1 wherein the second position is closer to the electrode than the first position.

4. The detector of claim 1 wherein the probe is immobilized on the electrode at a position distant from the redox moiety.

5. The detector of claims 1-4 wherein the electrode is capable of inducing redox events in the redox moiety.

6. The detector of claim 1 wherein the first configuration comprises internal hybridization between two regions in the polynucleotide probe.

7. The detector of claim 2 wherein the first configuration comprises internal hybridization between two regions in the polynucleotide probe.

8. The detector of claim 3 wherein the first configuration comprises internal hybridization between two regions in the polynucleotide probe.

9. The detector of claim 4 wherein the first configuration comprises internal hybridization between two regions in the polynucleotide probe.

10. The detector of claim 5 wherein the first configuration comprises internal hybridization between two regions in the polynucleotide probe.

11. The detector of claim 6 wherein the second configuration comprises a disrupted internal hybridization as the result of hybridization between a region in the polynucleotide probe and a complementary region in the polynucleotide.

12. The detector of claim 7 wherein the second configuration comprises a disrupted internal hybridization as the result of hybridization between a region in the polynucleotide probe and a complementary region in the polynucleotide.

13. The detector of claim 8 wherein the second configuration comprises a disrupted internal hybridization as the result of hybridization between a region in the polynucleotide probe and a complementary region in the polynucleotide.

14. The detector of claim 9 wherein the second configuration comprises a disrupted internal hybridization as the result of hybridization between a region in the polynucleotide probe and a complementary region in the polynucleotide.

15. The detector of claim 10 wherein the second configuration comprises a disrupted internal hybridization as the result of hybridization between a region in the polynucleotide probe and a complementary region in the polynucleotide.

16. The detector of claim 6 wherein the polynucleotide probe in its first configuration is a stem and hairpin configuration with the stem immobilized on the electrode and with the redox moiety attached to the end of the polynucleotide probe distal to the stem.

17. The detector of claim 11 wherein the polynucleotide probe in its first configuration is a stem and hairpin configuration with the stem immobilized on the electrode and with the redox moiety attached to the end of the polynucleotide probe distal to the stem.

18. The detector of claim 7 wherein the polynucleotide probe in its first configuration is a stem and hairpin configuration with the stem immobilized on the electrode and with the redox moiety attached to the end of the polynucleotide probe distal to the stem.

19. The detector of claim 12 wherein the polynucleotide probe in its first configuration is a stem and hairpin configuration with the stem immobilized on the electrode and with the redox moiety attached to the end of the polynucleotide probe distal to the stem.

20. The detector of claim 8 wherein the polynucleotide probe in its first configuration is a stem and hairpin configuration with the stem immobilized on the electrode and with the redox moiety attached to the end of the polynucleotide probe distal to the stem.

21. The detector of claim 13 wherein the polynucleotide probe in its first configuration is a stem and hairpin configuration with the stem immobilized on the electrode and with the redox moiety attached to the end of the polynucleotide probe distal to the stem.

22. The detector of claim 9 wherein the polynucleotide probe in its first configuration is a stem and hairpin configuration with the stem immobilized on the electrode and with the redox moiety attached to the end of the polynucleotide probe distal to the stem.

23. The detector of claim 14 wherein the polynucleotide probe in its first configuration is a stem and hairpin configuration with the stem immobilized on the electrode and with the redox moiety attached to the end of the polynucleotide probe distal to the stem.

24. The detector of claim 10 wherein the polynucleotide probe in its first configuration is a stem and hairpin configuration with the stem immobilized on the electrode and with the redox moiety attached to the end of the polynucleotide probe distal to the stem.

25. The detector of claim 15 wherein the polynucleotide probe in its first configuration is a stem and hairpin configuration with the stem immobilized on the electrode and with the redox moiety attached to the end of the polynucleotide probe distal to the stem.

26. The detector of claim 1 wherein the second configuration comprises internal hybridization between two regions in the polynucleotide probe.

27. The detector of claim 6 wherein the second configuration comprises internal hybridization between two regions in the polynucleotide probe.

28. The detector of claim 11 wherein the second configuration comprises internal hybridization between two regions in the polynucleotide probe.

29. The detector of claim 8 wherein the second configuration comprises internal hybridization between two regions in the polynucleotide probe.

30. The detector of claim 13 wherein the second configuration comprises internal hybridization between two regions in the polynucleotide probe.

31. The detector of claim 1 wherein the second configuration comprises a loop comprising a region of the target polynucleotide and a region of the polynucleotide probe.

32. The detector of claim 3 wherein the second configuration comprises a loop comprising a region of the target polynucleotide and a region of the polynucleotide probe.

33. The detector of claim 1 wherein the electrode comprises a metal.

34. The detector of claim 33 wherein the metal is gold.

35. The detector of claim 1 wherein the redox moiety is selected from the group consisting of ferrocene, viologen, methylene blue, anthroqunone, ethidium, bromide and daunomycin.

36. A detector for determining the presence of a target polynucleotide having a target nucleotide sequence said detector comprising an electrode capable of sensing redox events in a redox moiety and a polynucleotide probe comprising a first region, a second region, a third region, a fourth region and a fifth region,
the first region being immobilized on or proximate to the electrode,
the fifth region being bound to a redox moiety,
the second, third and fourth regions being present in the polynucleotide intermediate said first and fifth regions,
the second and fourth regions being capable of hybridizing one another and being spaced apart from one another by the third region.
the third region being of hairpin-forming length such that when the second and fourth regions hybridize one another a hairpin is formed and the fifth region with its bound redox moiety is brought into redox-detectable proxity to the electrode;
at least a portion of the second, third and fourth regions including a sequence capable of forming a target hybrid with the target polynucleotide, the formation of the target hybrid interfering with the hybridization between the second and fourth regions and interfering with the redox moiety being brought into redox-detectable proxity to the electrode.

37. The detector of claim 36 additionally comprising means for detecting electron transduction between the electrode and the redox moiety when the hairpin is formed.

38. The detector of claim 37 additionally comprising means for inducing electron transduction between the electrode and the redox moiety when the hairpin is formed.

39. The detector of claim 38 wherein the first region is at one end of the probe.

40. The detector of claim 38 wherein the fifth region is at one end of the probe.

41. The detector of claim 40 wherein the fifth region is at the second end of the probe.

42. The detector of claim 36 wherein the electrode comprises a metal.

43. The detector of claim 42 wherein the metal is gold.

44. The detector of claim 36 wherein the redox moiety is selected from the group consisting of ferrocene, viologen, methylene blue, anthroqunone, ethidium, bromide and daunomycin.

45. A detector for determining the presence of a target polynucleotide having a target nucleotide sequence said detector comprising an electrode capable of sensing redox events in a redox moiety and a polynucleotide probe comprising a first region, a second region and a third region,
the first region being immobilized or proximate to the electrode,
the third region being bound to a redox moiety,
the second region including a first nucleotide sequence which is complementary to and spaced apart from a second sequence with which it self hybridizes to form a first loop which positions the redox moiety a first distance from the electrode, said first nucleotide sequence also hybridizing with the target nucleotide sequence in the target polynucleotide, such hybridizing with the target nucleotide disrupting the first loop and permitting complementary nucleotide sequences in the second region to self hybridize to form a second loop which positions the redox moiety a second distance from the electrode.

46. The detector of claim 45 additionally comprising means for detecting electron transduction between the electrode and the redox moiety when the second loop is formed.

47. The detector of claim 46 additionally comprising means for inducing electron transduction between the electrode and the redox moiety when the loop is formed.

48. The detector of claim 47 wherein the first region is at one end of the probe.

49. The detector of claim 47 wherein the third region is at the second end of the probe.

50. The detector of claim 45 wherein the electrode comprises a metal.

51. The detector of claim 50 wherein the metal is gold.

52. The detector of claim 51 wherein the redox moiety is selected from the group consisting of ferrocene, viologen, methylene blue, anthroqunone, ethidium, bromide and daunomycin.

53. A method for detecting the presence of a target polynucleotide having a target nucleotide sequence in a sample comprising:
contacting the sample under polynucleotide hybridization conditions with the detector of claim 1 and sensing redox events in the redox moiety in the presence of the sample and redox events with the detector in the absence of the sample and,
correlating similarity in redox events between the two sensings with the absence of the target polynucleotide and a change in redox events with the presence of the target polynucleotide.

54. The method of claim 53 wherein the target polynucleotide is associated with an object and wherein the sensing of the presence of the target polynucleotide is correlated with the authenticity of the object.

55. A method for authenticating an object comprising:
obtaining a detector of claim 1
associating the object with the target polynucleotide of claim 1
sensing the presence of the target polynucleotide associated with the object; and
correlating the sensing of the presence of that target polynucleotide with the authenticity of the object.

56. The method of claim 54 or 55 wherein the sensing is carried out in the presence of masking DNA.
